(19) United States
(12) Patent Application Publication
(10) Pub. No.: US 2020/0337848 A1
(43) Pub. Date: Oct. 29, 2020

(54) METHOD FOR MANUFACTURING ARTIFICIAL CARTILAGE AND ARTIFICIAL CARTILAGE MANUFACTURED WITH THE METHOD

(71) Applicant: Sue-May KANG, Taipei City (TW)

(72) Inventor: Sue-May KANG, Taipei City (TW)

(21) Appl. No.: 16/928,780

(22) Filed: Jul. 14, 2020

Related U.S. Application Data
(63) Continuation of application No. 15/876,291, filed on Jan. 22, 2018.

(30) Foreign Application Priority Data
Jan. 25, 2017 (TW) 106124150

(51) Int. Cl. A61F 2/30

(52) U.S. Cl. CPC A61F 2/30756; A61F 2/30942; A61F 2002/30971; A61F 2002/3084; A61F 2002/30087; A61F 2/30771

(57) ABSTRACT

The present invention includes two methods for manufacturing an artificial cartilage and two types of artificial cartilage manufactured thereby, one of the said artificial cartilages can be utilized through implanting surgery fixed into an individual natural joint of an individual, and the other into an artificial joint of an individual joint of an individual before or during implanting surgery. The present invention is invented based on JOINT-ELECTRICITY THEORY created by the present inventor. After the said artificial cartilage is implanted, it can effectively react to the intra-articular dynamic pressure to continuously cause piezoelectricity effect for continuously generating Joint-Electricity, and to generate a sufficient amount of Joint-Electricity during daily living, so as to reduce pain, improve muscular strength, and speed the recovery of active motion ability after surgery.

### CROSS-REFERENCE TO RELATED APPLICATIONS

This application is a continuation-in-part of U.S. patent application Ser. No. 15/876,291, filed on Jan. 22, 2018 and entitled ARTIFICIAL CARTILAGE CAPABLE OF SUPPLEMENTING JOINT-ELECTRICITY, which claims the priority from Taiwan Patent Application No. TW106124150 filed on Jan. 25, 2017, the complete subject matter of both which are incorporated herein as reference.

### BACKGROUND OF THE INVENTION

### Field of the Invention

The present invention relates to artificial cartilage, and more particular to a method for manufacturing an artificial cartilage that continuously generates electricity in a joint, and an artificial cartilage manufactured with the method.

The present invention is based on JOINT-ELECTRICITY THEORY, which was created in 2010 and verified in 2011, both by the present inventor, Sue-May Kang. Based on such a theory, a healthy joint cartilage and an effective artificial cartilage must continuously generate Joint-Electricity (or, intra-articular electricity) in order to maintain the health of the joint, and normal muscle strength and motion of the related muscles of the said joint.

### Description of the Prior Art

Referring to FIG. 1, a joint in the human body is typically a junction between two bones G1 and G2. The joint includes cartilage C1, C2, locating at the end of each of the bones G1, G2, respectively. The cartilage in a joint is essentially composed of collagen fibers and has a smooth joint-surface. The cartilage C1, C2 is with its bottom end tightly attached to the corresponding end of bone, G1, G2, respectively. The cartilage C1, C2 provides a function of mitigating the pressure between bones. In other words, when compressed, the cartilage C1, C2 are deformed slightly and get thinner, and on release the pressure, the cartilage C1, C2 regain their thickness. Neither innervated, nor supplied nutrition with blood vessel, the cartilages stay in long term normal condition only when there is adequate joint exercise (those performed with proper bony alignment).

However, pathology may develop in human joints with varying causes. Particularly, those involving cartilage-damage demand medical treatments, and if which cannot obtain optimal results, may cause agonizing pains, decreasing intra-articular spaces, range of motion, and muscular strength, and developing severer motion difficulties. Further, the joint deformity consequently occurs such that the forces between the bones G1, G2 are transmitted in improper directions, would result in a vicious circle involving further lesions on the said cartilage, and, decreasing motor functions. If it cannot be improved through rehabilitative treatments, applicable interventions can include cartilage regeneration, as well as cartilage repair, or, cartilage replacement with an artificial cartilage, prosthetic cartilage, or the so-called biomimetic cartilage. More severe cases may require implanting an artificial joint in an attempt to relatively ameliorate the existing condition and reduce pain.

The present inventor discovered that the artificial cartilages implanted in the natural joints in the prior art serve only as a cushion between the adjacent ends of two bones G1, G2 in the joint, and do not generate Joint-Electricity, after being implanted in the human body. The artificial joints nowadays lack artificial cartilage and do not generate Joint-Electricity.

Since the known artificial cartilage and artificial joints do not generate Joint-Electricity to nurture neighboring tissues, following a surgical procedure, the parts of the human body and neighboring tissues are not supplied with sufficient Joint-Electricity and thus, result in severe pains, leading to the result that most patients need a long term and high dosage of painkillers. Further, there are difficulties in voluntary motions involving the post-operation joint. The patients suffer from further pains and difficulties during their motion therapies, and most patients need extra dosage of painkillers during their motion therapy sessions. Further, after their long term and painstaking motion therapies, there are minimal gains in muscular strength and voluntary motion ability and consequently slows down the recovery in their voluntary motor functions those they have expected.

Furthermore, these patients, without an effective artificial cartilage, there would be problems concerning a joint capsule suffering shortening and/or abnormality of tension, making the joint transfer force in an incorrect direction and amount during motion of the joint, and leading to that their artificial joints or artificial cartilages are prone to be soon worn-out (most of athletes cannot successfully resume their careers in sport after the said operations). High ratio of the post-operation joints requires to have repeated surgeries.

Thus, the major issue and goal of the present invention is to effectively solve the above problems by providing a method for manufacturing an artificial cartilage that is capable of continuously generating Joint-Electricity to achieve a purpose of supplementing Joint-Electricity to the said affected joint and its related muscles after surgery, in order to reduce pain, and improve motion function, and, thus, living quality of patients after implanting of artificial cartilage or artificial joint.

### BRIEF SUMMARY OF THE INVENTION

In order to overcome the problems of prior art, the present inventor, based on JOINT-ELECTRICITY THEORY that created by the present inventor, and years' study and research, develops a method for manufacturing an artificial cartilage that continuously generates Joint-Electricity (intra-articular electricity), and an artificial cartilage manufactured with the method.

The said method for manufacturing the artificial cartilage that continuously generate Joint-Electricity described in the present invention is based on JOINT-ELECTRICITY THEORY, which was created in 2010, and verified in 2011, both by the present inventor, Sue-May Kang. The parts of JOINT-ELECTRICITY THEORY related to the present invention are summarized as follows:

(1) A human joint can generate Joint-Electricity (intra-articular electricity, or electricity in a joint). The generation of Joint-Electricity is due to the cartilage (s) in a joint is with the property of piezoelectricity, and that it is constantly subjected to the dynamic pressure within the individual joint. Further, a joint can continuously generate Joint-Electricity only when the cartilage is constantly subjected to a dynamic pressure in an effective range (which is explained in the following). This is because the cartilage of a human joint is a piezoelectric material and has its own specific piezoelectric reactive range. The term “piezoelectric reactive range” used herein refers to a range of dynamic pressure that causes a piezoelectricity effect in the piezoelectric material. The reason that a normal joint can continuously generate Joint-Electricity is that the range of the intra-articular dynamic pressure always falls within the specific range that causes the piezoelectricity effect of the cartilage of the joint, and such a specific range is referred to as an effective range of dynamic pressure.

(2) Joint-Electricity generated in a joint can arrive and nurture the neighboring tissues of the joint. The said tissues include at least the joint-structure tissues (joint capsule, ligaments, and tendons) of the joint and its related muscles (those are connected to or overlapping the joint). With continuous generation of sufficient Joint-Electricity, the effects of nurture include keeping the length and tension of the joint-structure tissues normal, and keeping the related muscles in a ready condition for muscle contraction, so as to maintain the muscle strength and active motion in normal, given the nervous system is intact. The effect of nurture would be hindered if no sufficient Joint-Electricity has been generated for a long period of time.

(3) All skeletal muscles require to be nurtured by Joint-Electricity. (3-1) The joints defined in JOINT-ELECTRICITY THEORY include typical joints and untypical joints. The typical joints include movable joints, slightly movable joints, and non-movable joints, which have all been described in the related textbooks. The untypical joint is a term originating from JOINT-ELECTRICITY THEORY and bears a definition in the THEORY as follows: In a nontypical joint, the cartilage is located at a margin of a bone, but without the structure of a typical joint, and also can generate electricity when it is subjected to a dynamic pressure as a combination of a pull from the muscles connected to the joint and/or an external force externally applied to the joint. The electricity that they generate is also called Joint-Electricity. The sites of the said cartilages are called untypical joints and the untypical joints are not included in the indication-joints in the application of the present invention. (3-2) Based on JOINT-ELECTRICITY THEORY, the classification of the related muscles of the joints is as follows. A muscle having two ends that are each connected to one joint and is nurtured by the Joint-Electricity generated from the two joints is referred to as a two-joint-nurturing muscle. A muscle that has just one end connected to a joint and is nurtured by Joint-Electricity generated from the joint is referred to as a one-joint-nurturing muscle. A muscle that has two ends both located at the sites other than a joint and receives Joint-Electricity generated from nearby joint(s) is referred to as a neighbor-nurturing muscle. There are few neighbor-nurturing muscles in the human body, and most of the muscles are connected to one or two joints. Thus, the availability of the cartilage in a joint that can normally or continuously generate Joint-Electricity is very important to the joint-structure tissues and the related muscles of the joint.

(4) Joint-Electricity generated from the neighboring joints of the affected joint, when in an amount large enough, can, in some degree, supplement the wanted nurturing requirement of the neighboring tissues of the affected joint. Since the artificial cartilage and artificial joint in the prior art cannot generate Joint-Electricity, the motion therapy applied to a joint following surgery must involve effective training of the nearest neighboring joints that are healthy in order to generate large enough Joint-Electricity to, in some degree, supplement the wanted Joint-Electricity for the requirement of the operated joint. However, only few cases can achieve the success.

(5) Dynamic pressure within the typical joint comes from two major sources. All typical joints, having cartilages locate between multiple bones and joint capsules or ligaments outside the cartilages, would generate an intra-articular reaction force in response to the force acting on the joint. This is one of the major sources of the intra-articular dynamic pressure. The other major source of intra-articular dynamic pressure primarily comes from blood pulses, breathing rhythm, and the motions of viscera. This kind of dynamic pressure is called vital-organ mechanism, and is especially dominant when the human body is sleeping. The instant dynamic pressure for a typical joint is the instant sum of the intra-articular reaction force and dynamic pressure that comes from vital-organ mechanism. When the range of the intra-articular dynamic pressure is in the range of piezoelectric reactivity of the cartilage, the cartilage of the joint would generate Joint-Electricity due to the piezoelectricity effect.

(6) According to JOINT-ELECTRICITY THEORY, the alignment of the joint-structure of a joint should be correct in order to have every part of the cartilage in the joint receive a dynamic pressure in a correct direction during the joint motions in all directions. The correct alignment of a joint structure requires not only the joint-structure (bones, capsule, ligament, tendon, and joint space) being corrected to be totally correct with proper surgery (explained in the following), but also the shape of the artificial cartilage satisfying the requirement for correctness of the joint-structure, in order not to interfere with the intra-articular dynamic pressure of the joint in a correct direction and normal range. The so-called proper surgery as mentioned above refers to the surgery for implanting an artificial cartilage or an artificial joint, and during the said surgery, it is simultaneously obtained the correct alignment of the joint, the limb, and the whole body of the subject, and the fully recovered (or corrected) normal structure of the joint-structure tissues (capsule, ligament, and tendon). The later one is obtained through the methods of first, reconstructing the related tissues, including reserving as much as possible the related tissues, and if necessary, repairing, or, supplementing the related tissues, second, reconstructing the normal joint space, with a size the same as what should normally be in each motion direction, and reconstructing the adequate joint fluid in both amount and quality, wherein, the joint fluid or artificial joint fluid is appropriately supplemented as required. The patient's joint, once treated with such proper surgery, would allow the intra-articular dynamic pressure in every part of the surgery-treated joint to be correct in both direction and normal range. It is noted that the artificial cartilage manufactured with the method of the present invention can be fully effective after implantation of it when the said implantation is with the proper surgery, yet such surgery is similar to the known processes that have been adopted to implant artificial cartilage of any other kinds and such surgery is well known in the art, so that the surgery or any other surgery is not included in the scope of the present invention. Unless necessary, in the disclosure of the present invention, no further detail concerning the method and contents of the surgery will be provided, and it will be simply referred to as “surgery” or “proper surgery”.

The present invention includes two methods for manufacturing an artificial cartilage, and two types of artificial cartilage manufactured with each of the said methods, respectively. One of the said artificial cartilages can be utilized through implanting surgery fixed into an individual natural joint of an individual, and the other into an artificial joint of an individual joint before or during implanting surgery. The said manufacturing method in the present invention is invented based on Joint-Electricity THEORY created by the present inventor. After the surgery implanting it into an individual joint of an individual, or together with an artificial joint into an individual, it can effectively react to the intra-articular dynamic pressure to continuously cause piezoelectricity effect for continuously generating Joint-Electricity. Further, with the said special designed manufacturing method in the present invention, it can generate a sufficient amount of Joint-Electricity in daily living. Thus, it can effectively reduce pain, improve muscular strength, and speed the recovery of active motion ability after surgery.

The first method for manufacturing an artificial cartilage of the present invention is disclosed in the following. The said method is for manufacturing an artificial cartilage that is to be implanted to an individual joint (an individual natural joint) for an individual. The method comprises: making continuous measurement on the intra-articular pressure of the contralateral joint of the said individual joint during daily living for deciding the range of the said intra-articular pressure in order to make an estimation of the range of dynamic pressure inside the contralateral joint that acts on the joint-surface of the cartilage of the contralateral joint, and, based on the said range, making an estimation of the range of intra-articular dynamic pressure that acts on the joint-surface of the said artificial cartilage after the said artificial cartilage is implanted; (the method of the previous step being performed without any particular order in relation to the method of the next step); making continuous measurement on the level of Joint-Electricity generated by the contralateral joint of the said individual joint during daily living for deciding the range of the said level, and, based on the said range, making an estimation of the range of level of Joint-Electricity that is required to be generated by the said artificial cartilage after the said artificial cartilage is implanted into the said individual joint, simply called range of level of Joint-Electricity required; searching and finding a piezoelectric material according to the said range of intra-articular dynamic pressure, and the said range of level of Joint-Electricity required, wherein the piezoelectric material has the material properties at least of: the range of piezoelectric reactivity of the said piezoelectric material must be wider than the said range of intra-articular dynamic pressure, and the range of level of electricity the said piezoelectric material generates under the said range of intra-articular dynamic pressure must be wider than the said range of level of Joint-Electricity required; and forming the said individual artificial cartilage of the said individual joint with the said piezoelectric material.

In the above method, the said range of piezoelectric reactivity of a piezoelectric material is defined as the range of dynamic pressure that the said piezoelectric material would react to it to effectively cause a piezoelectricity effect.

In the above method, the said continuous measurement is made during daily living, wherein, the said daily living is defined including the activities lightest to heaviest performed by the said individual, such as resting, (or, sleeping), general daily living activities, and the heaviest sport that the individual can perform at the time deciding to manufacture the said artificial cartilage.

In the above method, the said estimation of the range of intra-articulate dynamic pressure of the said individual joint after the implanting surgery also includes the increase of the said range resulting from the improvement of the motion ability of the joint due to the implantation of the artificial cartilage of the present invention in the said individual joint. Similarly, the said estimation of the range of level of Joint-Electricity required for the said individual joint after implanting surgery also includes the increase of the said range resulting from the improvement of the motion ability of the joint due to the implantation of the artificial cartilage of the present invention in the said individual joint.

In the above method, the piezoelectric material that is so found and meets the above requirements must be also with a material property of biocompatibility. Material property of biocompatibility is an essential requirement for all medical or bioengineering devices implanted into the human body. This part of method is known in the related art, and thus, is not created by the present invention.

The method disclosed at the above is for manufacturing an artificial cartilage that is to be implanted to an individual joint (individual natural joint) for an individual, wherein the method for forming the said artificial cartilage with the said piezoelectric material includes, the said piezoelectric material being formed into the said artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface is further made to be extremely smooth as of nanometer scale.

In the above method, the said structure, shape, and size of the said individual joint as mentioned above, if necessary, also include the structure and shape of the individual joint that is obtained after the recovering and correcting processes during the said proper surgery for implanting it.

In one embodiment, the method for forming the artificial cartilage that is to be implanted to an individual natural joint of an individual can include: making the said piezoelectric material into the fibers having a diameter of nanometer size and forming the said fibers into an artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface has become extremely smooth as of nanometer scale.

In another embodiment, the method for forming the artificial cartilage that is to be implanted to an individual natural joint of an individual can include forming the said piezoelectric material into an artificial cartilage having the individualized shape of the said individual artificial cartilage according to the structure, shape, and size of the said individual joint, wherein the said individualized shape of the said artificial cartilage has a smooth joint-surface, and then the said smooth joint-surface being further coated with the said piezoelectric material that has been processed to be of nanometer size for making the said joint-surface extremely smooth as of nanometer scale.

In another embodiment, the method for forming the artificial cartilage that is to be implanted to an individual natural joint of an individual can include: processing the said piezoelectric material into the particles of nanometer size, and forming the said particles into an artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface after this method has become extremely smooth as of nanometer scale.

The present invention also mentions an artificial cartilage. The said artificial cartilage is made with the method disclosed above, and is that to be utilized in implanting into the said individual natural joint of an individual.

Wherein, the method for utilizing the said artificial cartilage is through implanting surgery, especially the proper surgery that has been described in [0016]. The so-called proper surgery is known to surgeons skilled in artificial cartilage implanting surgery, and is not included in the scope of patent protection of the present invention.

The present invention also provides second method for manufacturing an artificial cartilage. The said method is for manufacturing an artificial cartilage that is to be implanted or fixed to an individual artificial joint for an individual joint of an individual. The method comprises: making continuous measurement on the intra-articular pressure of the contralateral joint of the said individual joint during daily living for a range of the said intra-articular pressure in order to make an estimation of a range of dynamic pressure inside the said contralateral joint that acts on the joint-surface of cartilage of the said contralateral joint, based on the said range, making an estimation of a range of intra-articular dynamic pressure that acts on the joint-surface of the said artificial cartilage after the said artificial cartilage has been implanted to the said individual joint, and applying a force transmission correction parameter to correct the said range of intra-articular dynamic pressure to obtain a corrected range of intra-articular dynamic pressure, which is the estimation of the range of dynamic pressure inside the said individual artificial joint that acts on the joint-surface of the said artificial cartilage after the said artificial joint is implanted, wherein the force transmission correction parameter is determined according to the structure and material of the said individual artificial joint; making continuous measurement on the level of Joint-Electricity generated by the contralateral joint of the said individual joint during daily living for a range of the said level, and, based on the said range, making an estimation of a range of the level of Joint-Electricity required to be generated by the said individual joint after the said individual artificial cartilage is implanted into the said individual artificial joint, wherein the said range is simply called range of the level of Joint-Electricity required; (the two previous methods being performed without any specific order); searching and finding a piezoelectric material according to the said corrected range of intra-articular dynamic pressure and said range of the level of Joint-Electricity required of the said individual joint, wherein the piezoelectric material has the material properties at least of: a range of piezoelectric reactivity of the piezoelectric material must be wider than the said corrected range of intra-articular dynamic pressure, and a range of the level of electricity generated by the said piezoelectric material under the said corrected range of intra-articular dynamic pressure must be wider than the said range of the level of Joint-Electricity required; and forming the said individual artificial cartilage for the said individual artificial joint of the said individual joint with the said piezoelectric material.

In the above method, the said continuous measurement is made during daily living, wherein, the said daily living is defined including the activities lightest to heaviest performed by the said individual, such as resting, (or, sleeping), general daily living activities, and the heaviest sport that the individual can perform at the time deciding to manufacture the said artificial cartilage.

In the above method, the estimation of the range of intra-articulate dynamic pressure that acts on the joint-surface of the said artificial cartilage after it has been implanted also includes the increase of the said range resulting from the improvement of the motion ability of the joint due to the implantation of the artificial joint that has an artificial cartilage of the present invention manufactured for the said individual joint.

In the above method, the estimation of the range of the level of Joint-Electricity required for the said artificial cartilage after it has been implanted also includes the increase of it resulting from the improvement of the motion ability of the joint due to the implantation of the artificial joint that has an artificial cartilage of the present invention manufactured for the said individual joint.

In the above method, the piezoelectric material that is so found and meets the above requirements must be also with a material property of biocompatibility. Material property of biocompatibility is an essential requirement for all medical or bioengineering devices implanted into the human body. This part of method is known in the related art, and thus, is not created by the present invention.

In an embodiment of the method for manufacturing an artificial cartilage of the present invention that is to be implanted or fixed to an individual artificial joint for an individual joint of an individual, the method for forming the said artificial cartilage with the said piezoelectric material includes, the said piezoelectric material being formed into the said artificial cartilage having the individualized shape of the said individual artificial cartilage according to the structure, shape, and size of the said individual artificial joint of the said individual joint, wherein the said individualized shape of the said artificial cartilage has a smooth joint-surface, and wherein, the said smooth joint-surface is further made to be extremely smooth as of nanometer scale.

In one embodiment, the method for forming the artificial cartilage that is to be implanted to an individual artificial joint for an individual joint of an individual can include: processing the said piezoelectric material into the fibers having a diameter of nanometer size, and forming the said fibers into the said artificial cartilage having the individualized shape of the said individual artificial cartilage according to the structure, shape, and size of the said individual artificial joint, wherein the said individualized shape of the said artificial cartilage has a smooth joint-surface, and through this method, an extremely smooth joint-surface as of nanometer scale is also obtained.

In another embodiment, the method for forming the artificial cartilage that is to be implanted to an individual artificial joint for an individual joint of an individual can include: forming the said piezoelectric material into the artificial cartilage having the individualized shape of the said individual artificial cartilage according to the structure, shape, and size of the said individual artificial joint, wherein the said individualized shape of the said artificial cartilage has a smooth joint-surface, and further coating the said piezoelectric material that has been made to be of nanometer size on the said smooth joint-surface in order to have it as smooth as of nanometer scale.

In another embodiment, the method for forming the artificial cartilage that is to be implanted to an individual artificial joint for an individual joint of an individual can include processing the said piezoelectric material into the particles of nanometer size, and forming the said particles into an artificial cartilage having the individualized shape of the said individual artificial cartilage according to the structure, shape, and size of the said individual artificial joint, wherein the said individualized shape of the said artificial cartilage has a smooth joint-surface, and wherein, the said process has made the said smooth joint-surface as smooth as of nanometer scale.

Optionally, according to the present invention, the forming method of an artificial cartilage for an individual artificial joint of an individual joint for an individual can be the following method: making the said piezoelectric material to be of nanometer size, and carrying out coating with the said nanometer-size material in multiple layers on a primary joint-surface of the said individual artificial joint to form the individualized shape of the said artificial cartilage according to the structure, shape, and size of the said individual artificial joint, wherein the said individualized shape of the artificial cartilage has a smooth joint-surface. Wherein, the said process has simultaneously made an extremely smooth joint-surface as of nanometer scale, forming the said artificial cartilage in its own individualized shape, and fixing the artificial cartilage to the said individual artificial joint. This method can only be applied before the surgery of implanting the said artificial joint.

The term of primary joint-surface as used herein refers to surfaces that are moved toward each other during joint motion in an artificial joint or in a natural joint at the site of an end of a bone to which a cartilage is connected to or an artificial cartilage is fixed to. However, the primary joint-surface of an artificial joint can be different in shape from that of a natural joint.

The present invention also mentions one artificial cartilage. The said artificial cartilage is made with the second method that stated above for manufacturing an artificial cartilage according to JOINT-ELECTRICITY THEORY, and is to be implanted to an individual artificial joint of an individual joint of an individual.

The method for utilizing the said artificial cartilage is through implanting surgery, especially the proper surgery that has been described in [0016] to implant the said individual artificial joint that has the said artificial cartilage. The so-called proper surgery is known to surgeons skilled in the surgeries those are for implanting artificial joint, and is not included in the scope of patent protection of the present invention.

The method for manufacturing an artificial cartilage, and the artificial cartilage so manufactured according to the said method in the present invention are both based on JOINT-ELECTRICITY THEORY created by the present inventor. After the said artificial cartilage has been implanted to the said individual joint that has been measured and the data of which is based on for manufacturing it, it would generate Joint-Electricity, in a continuous manner, and in a sufficient level, during the use thereof, providing improvement on both the joint health, and motion ability of the said individual joint. Such effects are the effects those are not provided in the artificial cartilages and artificial joints of the prior art, indicating the present invention is effective in remedying the drawbacks existing in the artificial cartilages and artificial joints of the prior art.

The present invention provides the methods for manufacturing artificial cartilages those either to be implanted to a natural joint, or, an artificial joint. The artificial cartilage so manufactured provide an effect the same as that of a natural cartilage, because they are made based on that a healthy joint-cartilage of a human body exhibits a piezoelectricity property, and cartilages of different joints exhibit different ranges of piezoelectric reactivity. Thus, the manufacturing method includes first making an estimation a range of intra-articular dynamic pressure in an individual joint, based on which, searching is made for a piezoelectric material that has a range of piezoelectric reactivity wider than the said range of intra-articular dynamic pressure to ensure the artificial cartilage so manufactured can always be effectively reacted to the dynamic pressure inside the joint to cause piezoelectricity effect in order to continuously supply Joint-Electricity. Further, the method also includes making an estimation of a range of the level of Joint-Electricity that is required to be generated by the said individual joint before finding the said piezoelectric material. The said range thus is also the required condition for searching the said piezoelectric material. The said piezoelectric material must also fulfill that it has a range of the level of electricity generated under the said range of intra-articular dynamic pressure thereby being wider than the said range of the level of Joint-Electricity required in order to ensure the artificial cartilage so manufactured, after implantation into the said individual joint or the artificial joint of the said individual joint, generates a sufficient amount of Joint-Electricity in relation to the requirement of the said individual joint.

Further, since the structure of an artificial joint must include metal and plastics, the transmission rate of force would be affected. Thus, the range of intra-articular dynamic pressure of the artificial joint would be different from that of a natural joint. Thus, in the method for manufacturing an artificial cartilage of the present invention that is to be implanted in an artificial joint, the range of intra-articular dynamic pressure that is estimated from the natural contralateral joint has been subjected to a correction according to the structure and material of the said individual artificial joint. This method ensures that after it is implanted, accompanied with the said artificial joint, into the said individual joint, it is capable of continuously generating Joint-Electricity.

Thus, compared to the artificial cartilage in the prior art which only functions as a cushion, and the artificial joint in the prior art which lacks an artificial cartilage, or lacks an artificial cartilage that could generate Joint-Electricity, an artificial cartilage manufactured with the manufacturing method created based on JOINT-ELECTRICITY THEORY, after implantation through surgery to the said individual joint (natural joint, or, artificial joint), may continuously generate Joint-Electricity to achieve an effect of supplementing Joint-Electricity, so as to effectively decrease the occurrence of pain of the joint following surgery, massively reduce the amount of pain killer, increase the muscular strength, and improve motion ability, thereby make active motion of the joint easy after surgery, increase the recovery speed of active motion ability, accelerate the recovery of the motion function of the joint following surgery, and effectively improve daily living quality of the patient after surgery. Further, Joint-Electricity generated by the artificial cartilage also help to extend the service life of the artificial cartilage or the artificial joint.

### BRIEF DESCRIPTION OF THE DRAWINGS

FIG. 1 is a schematic view showing the sagittal plane of a natural joint and cartilage of a human body;

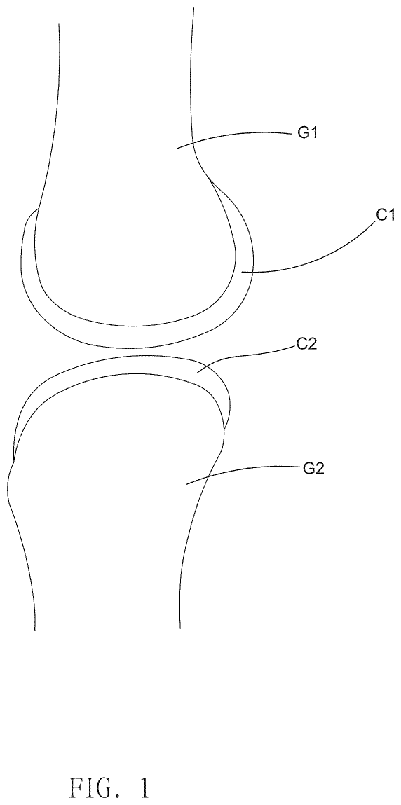

FIG. 2A is a flow chart of the first method for manufacturing an artificial cartilage according to the present invention;

FIG. 2B is showing multiple embodiments of the methods of forming the said artificial cartilage in the first method for manufacturing an artificial cartilage according to the present invention;

FIG. 2C is a flow chart of the second method for manufacturing an artificial cartilage according to the present invention;

FIG. 2D is showing multiple embodiments of the methods of forming the said artificial cartilage in the second method for manufacturing an artificial cartilage according to the present invention;

FIG. 3A is a schematic view showing the sagittal plane of an embodiment of the artificial cartilage manufactured by the first method at after it is implanted to a human natural joint;

FIG. 3B is a schematic view showing the sagittal plane of an embodiment of the artificial cartilage manufactured by the second method at after it is implanted to an individual artificial joint of an individual joint;

FIG. 4A is a schematic view showing the sagittal plane of an artificial cartilage formed according to method N41a of FIG. 2B that follows FIG. 2A in the method for manufacturing an artificial cartilage according to the present invention;

FIG. 4B is a schematic view showing the sagittal plane of an artificial cartilage formed according to method N41b of FIG. 2B that follows FIG. 2A in the method for manufacturing an artificial cartilage according to the present invention;

FIG. 4C is a schematic view showing the sagittal plane of an artificial cartilage formed according to method N41c of FIG. 2B that follows FIG. 2A in the method for manufacturing an artificial cartilage according to the present invention;

FIG. 5A is a schematic view showing the sagittal plane of an artificial cartilage formed according to method A41a of FIG. 2D that follows FIG. 2C in the method for manufacturing an artificial cartilage according to the present invention;

FIG. 5B is a schematic view showing the sagittal plane of an artificial cartilage formed according to method A41b of FIG. 2D that follows FIG. 2C in the method for manufacturing an artificial cartilage according to the present invention;

FIG. 5C is a schematic view showing the sagittal plane of an artificial cartilage formed according to method A41c of FIG. 2D that follows FIG. 2C in the method for manufacturing an artificial cartilage according to the present invention; and

FIG. 5D is a schematic view showing the sagittal plane of an artificial cartilage formed according to method A41d of FIG. 2D that follows FIG. 2C in the method for manufacturing an artificial cartilage according to the present invention.

### DETAILED DESCRIPTION OF THE INVENTION

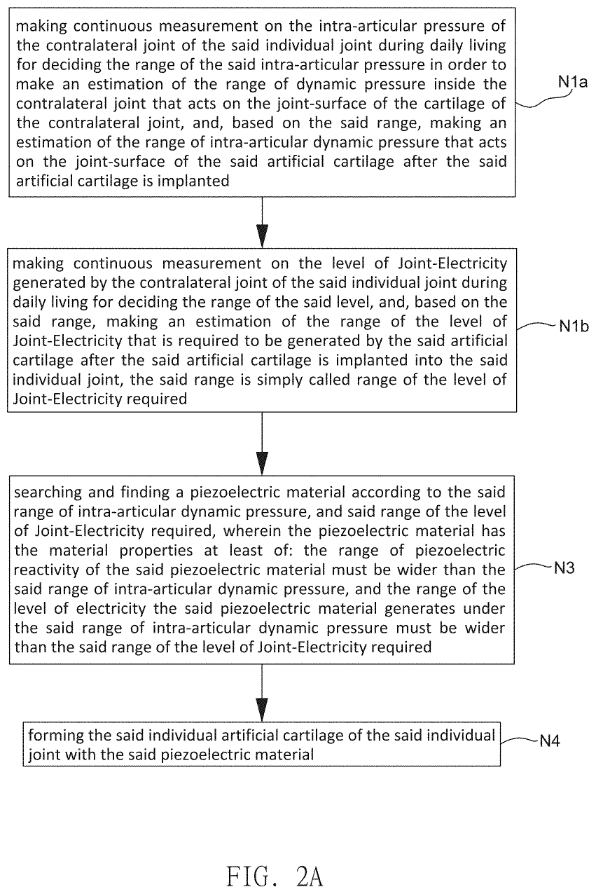

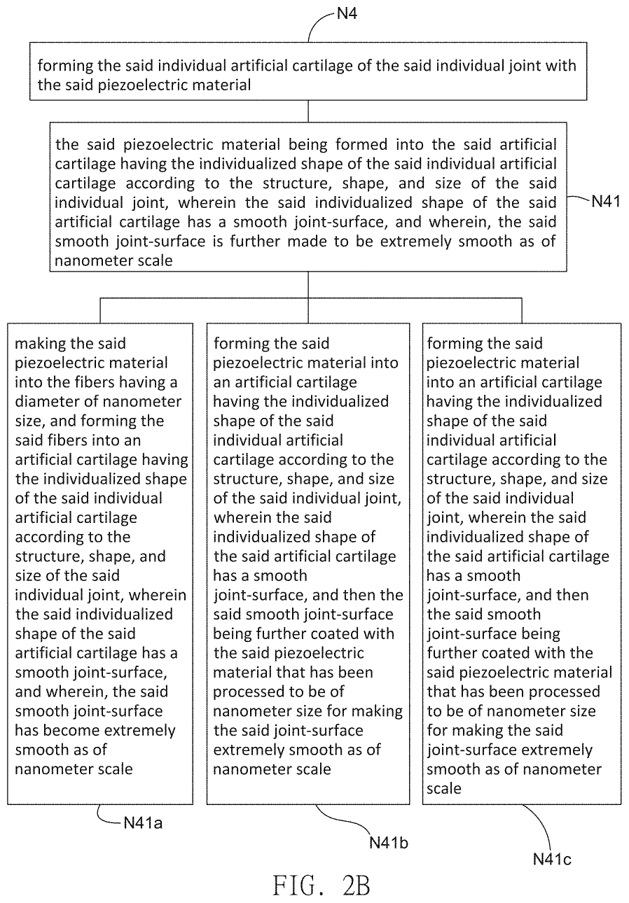

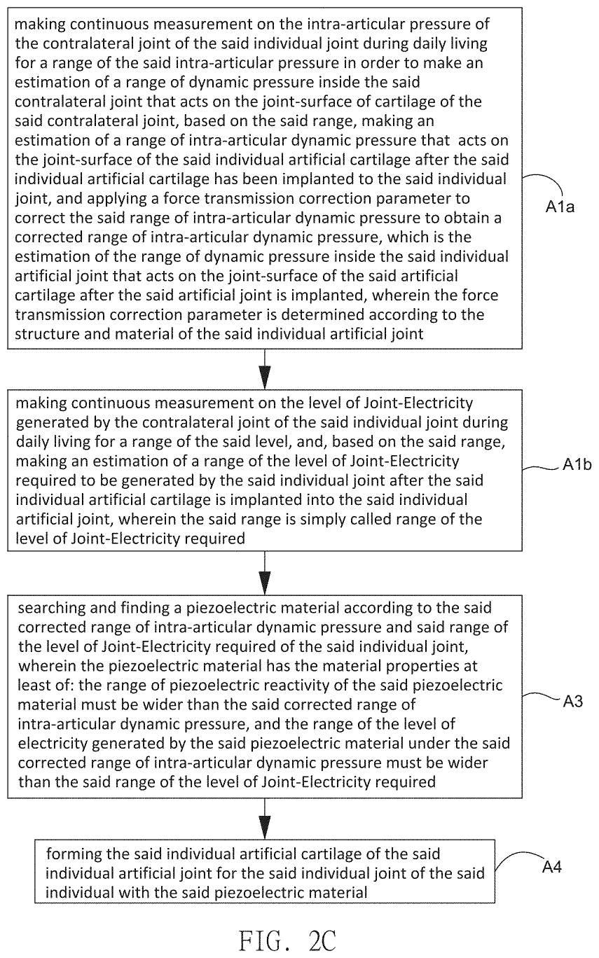

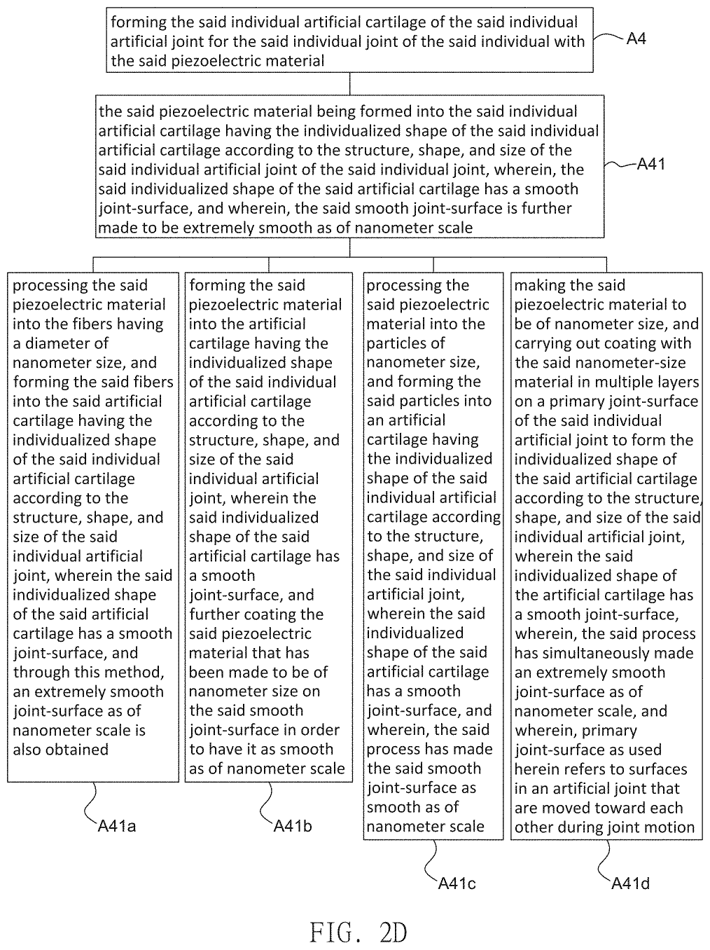

Embodiments of the present invention will be described below with reference to FIGS. 2A-5D. The description is not made to limit the way of embodying the present invention, and is rather provided as an example of embodying the present invention.

Firstly, referring to FIG. 2A, the drawing is a flow chart of a first method for manufacturing an artificial cartilage that is to be utilized through implanting into an individual joint (a natural individual joint) according to the present invention.

First, Method N1a and Method N1b are performed, and the sequence of performing the two is not specified.

Method N1a is described in the following: making continuous measurement on the intra-articular pressure of the contralateral joint of the said individual joint during daily living for deciding the range of it in order to make an estimation of the range of dynamic pressure inside the contralateral joint that acts on the joint-surface of the cartilage of the contralateral joint, and, based on the said range, making an estimation of the range of intra-articular dynamic pressure that acts on the joint-surface of the said artificial cartilage after it is implanted; (the method of the previous step being performed without any particular order in relation to the method of the next step).

In the embodiment, a method for making the estimation of the range of intra-articulate dynamic pressure that acts on the joint-surface of the artificial cartilage after the surgery also includes the increase of the said range resulting from the improvement of the motion ability of the joint due to the implantation of the artificial cartilage of the present invention in the said individual joint.

Method N1b is described in the following: making continuous measurement on the level of Joint-Electricity generated by the contralateral joint of the said individual joint during daily living for deciding the range of it, and, based on the said range, making an estimation of the range of the level of Joint-Electricity that is required to be generated by the said artificial cartilage after it is implanted into the said individual joint, the said range is simply called range of the level of Joint-Electricity required.

In the embodiment, a method for making the estimation of the range of the level of Joint-Electricity required for the said individual joint also includes the increase of the said range resulting from the improvement of the motion ability of the joint due to the implantation of the artificial cartilage of the present invention in the said individual joint.

In the above method, the said continuous measurement is made during daily living, wherein, the said daily living is defined including the activities lightest to heaviest performed by the said individual, such as resting, (or, sleeping), general daily living activities, and the heaviest sport that the individual can perform at the time deciding to manufacture the said artificial cartilage.

Method N3 is next performed, and the method is as following: searching and finding a piezoelectric material according to the said range of intra-articular dynamic pressure, and said range of the level of Joint-Electricity required, wherein the piezoelectric material has the material properties at least of: the range of piezoelectric reactivity of the said piezoelectric material must be wider than the said range of intra-articular dynamic pressure, and the range of the level of electricity the said piezoelectric material generates under the said range of intra-articular dynamic pressure must be wider than the said range of the level of Joint-Electricity required.

The term “range of piezoelectric reactivity of a piezoelectric material” as used herein refers to a range of dynamic pressure that the piezoelectric material would react to it to cause piezoelectricity effect.

More essentially, the piezoelectric material that is so found and meets the above requirement must be also having the material property of biocompatibility, so that the artificial cartilage does not cause harm to the human body and will not be rejected by the human body. The material property of biocompatibility is an essential requirement for all medical or bioengineering devices implanted into the human body. This method is known in the related art, and thus, is not created by the present invention.

Then, Method N4 is performed: forming said individual artificial cartilage of the said individual joint with the said piezoelectric material.

In the present invention, the said individual artificial cartilage is manufactured following the measurements and estimations of the data those are related to the said individual joint of an individual. Thus, the said individual artificial cartilage is manufactured aiming the said individual joint for the said individual, and being based on the said individual joint for the said individual.

Referring to FIG. 2B, the artificial cartilage forming method N4 has the embodiment N41: the method for forming the said artificial cartilage with the said piezoelectric material includes, the said piezoelectric material is formed into the said artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface is further made to be extremely smooth as of nanometer scale.

The structure and shape of the individual joint as mentioned above also include the structure and shape of the individual joint obtained after the repair and/or correcting during surgery.

The said individualized shape of the individual artificial cartilage according to the structure and shape of the individual joint of the individual as mentioned above is for the purpose of having the artificial cartilage not interfering with every part of the artificial cartilage receiving a dynamic pressure in a correct direction and a normal range after the artificial cartilage of the present invention has been implanted in the individual joint with the proper surgery. The said proper surgery has been defined in paragraph [0016].

The joint-surface of nanometer scale as mentioned above helps reduce potential mechanical abrasion inside the joint, allowing the artificial cartilage not to easily wear out during use thereof.

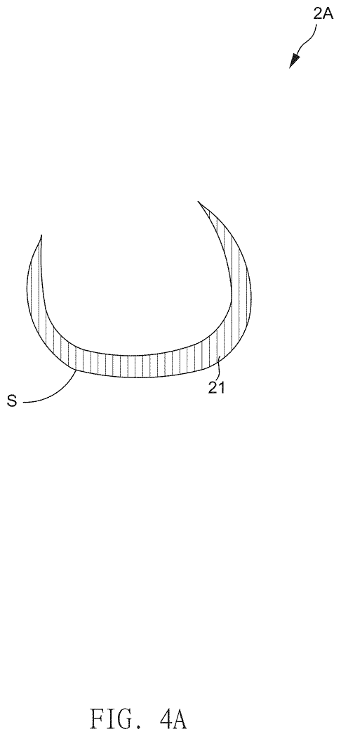

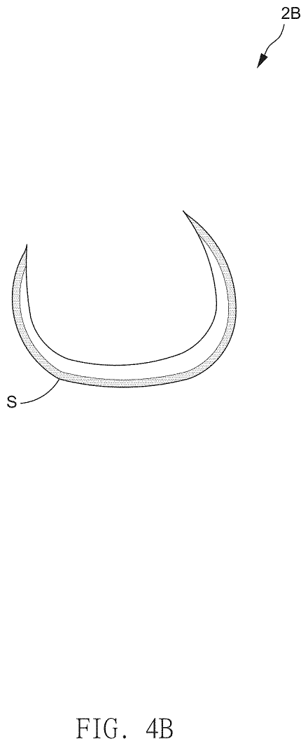

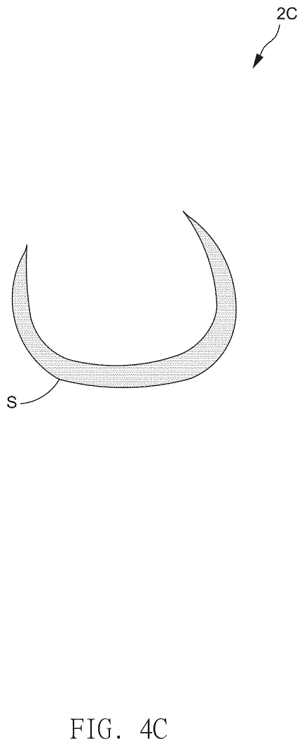

As shown in FIG. 2B, the artificial cartilage forming method N41 can be Method N41a: making the said piezoelectric material into the fibers 21 having a diameter of nanometer size, and forming the said fibers 21 into an artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface has become extremely smooth as of nanometer scale. The artificial cartilage 2A so manufactured is schematically illustrated in its sagittal plane in FIG. 4A, and the artificial cartilage 2A has a joint-surface S as smooth as of nanometer scale.

Optionally, as shown in FIG. 2B, the artificial cartilage forming method N41 can be Method N41b: forming the said piezoelectric material into an artificial cartilage having the individualized shape of the said individual artificial cartilage according to the structure, shape, and size of the said individual joint, wherein the said individualized shape of the said artificial cartilage has a smooth joint-surface, and then the said smooth joint-surface is further coated with the said piezoelectric material that has been processed to be of nanometer size for making the said joint-surface extremely smooth as of nanometer scale. The artificial cartilage 2B so manufactured is schematically illustrated in the sagittal plane in FIG. 4B, and the artificial cartilage 2B has a joint-surface S as smooth as of nanometer scale.

Optionally, as shown in FIG. 2B, the artificial cartilage forming method N41c can alternatively include Method N41c: processing the said piezoelectric material into the particles of nanometer size, and forming the said particles into an artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface after this method has become extremely smooth as of nanometer scale. The artificial cartilage 2C so manufactured is schematically illustrated in the sagittal plane in FIG. 4C, and the artificial cartilage 2C has a joint-surface S as smooth as of nanometer scale.

The present invention also claimed an artificial cartilage, which is manufactured by the method described above (as shown in the methods described in FIGS. 2A and 2B) and is referred to as the first method of manufacturing an artificial cartilage of the present invention. The said artificial cartilage is that can be utilized through implanting into an individual natural joint.

In the present invention, the said individual artificial cartilage is manufactured following the measurements and estimations of the data those are related to the said individual joint. Thus, the individual artificial cartilage is manufactured aiming the said individual joint, and based on the said individual joint.

Wherein, the method for utilizing the said artificial cartilage is through implanting surgery, especially the proper surgery that has been described in [0016]. The so-called proper surgery is known to surgeons skilled in artificial cartilage implanting surgery, and is not included in the scope of patent protection of the present invention.

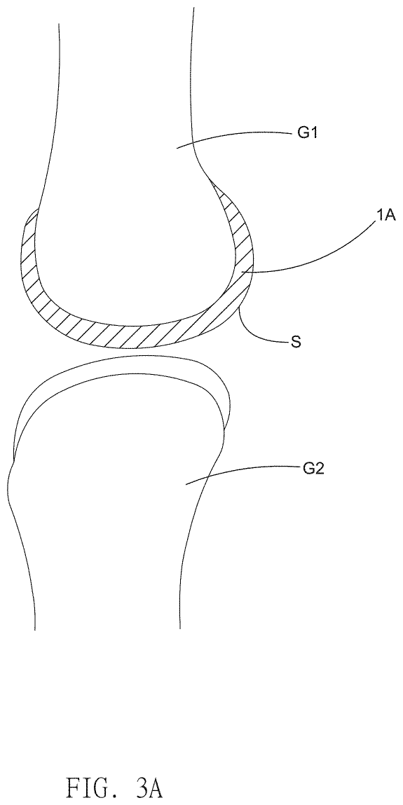

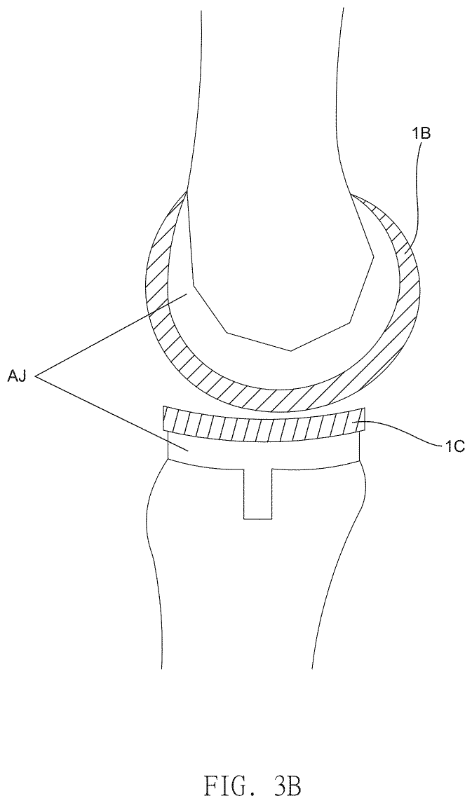

The artificial cartilage 1A so manufactured can be utilized to implant, through surgery, into an individual natural joint of an individual, as shown in FIG. 3A. After implantation, the artificial cartilage could continuously generate a sufficient amount of Joint-Electricity, similar to a healthy cartilage.

However, FIG. 3A is a schematic view showing one individual natural joint only. The said individual joint is not limited to any specific joint. The artificial cartilage of the present invention can be any individual cartilage suit to an individual joint of an individual.

Referring to FIG. 2C, the drawing is a flow chart of the second method for manufacturing an artificial cartilage according to the present invention. The method is for manufacturing an individual artificial cartilage for an individual artificial joint of an individual joint of an individual that can be utilized through implanting or fixing to the said individual artificial joint for the said individual joint of the said individual.

First, Method A1a and Method A1b are performed, and it is possible to first perform Method A1a and then perform Method A1b; or alternatively, Method A1b is performed first and then Method A1a is performed.

Ala is described in the following: making continuous measurement on the intra-articular pressure of the contralateral joint of the said individual joint during daily living for a range of it in order to make an estimation of a range of dynamic pressure inside the said contralateral joint that acts on the joint-surface of cartilage of the said contralateral joint, based on the said range, making an estimation of a range of intra-articular dynamic pressure that acts on the joint-surface of the said artificial cartilage after it has been implanted to the said individual artificial joint, and applying a force transmission correction parameter to correct the said range of intra-articular dynamic pressure to obtain a corrected range of intra-articular dynamic pressure, which is the estimation of the range of dynamic pressure inside the said individual artificial joint that acts on the joint-surface of the said artificial cartilage after the said artificial joint is implanted into the site of the said individual joint of the said individual.

In the above method, the force transmission correction parameter is determined according to the structure and material of the said individual artificial joint of the said individual joint of the said individual. This is because the structure of the artificial joint includes metal and plastics and would affect the transmission rate of force, and would also affect the range of dynamic pressure inside the artificial joint. The said correction parameter must be calculated according to different structure and material of each different artificial joint, and such a calculation method is known to those skilled in the art.

In the embodiment, the method for making the estimation of the range of intra-articulate dynamic pressure that acts on the joint-surface of the artificial cartilage of the said individual joint also includes the increase of the said range resulting from the improvement of the motion ability of the joint due to the implantation of the artificial joint that has an artificial cartilage of the present invention manufactured for the said individual joint.

A method of performing Method A1b is: making continuous measurement on the level of Joint-Electricity generated by the contralateral joint of the said individual joint during daily living for a range of it, and, based on the said range, making an estimation of a range of the level of Joint-Electricity required to be generated by the said individual joint after the said individual artificial cartilage is implanted into the said individual artificial joint through a surgery, the said range is simply called range of the level of Joint-Electricity required. Method A1b is similar to Method N1b.

In the embodiment, a method for making the estimation of the range of the level of Joint-Electricity required for the said individual joint also includes the increase of the said range resulting from the improvement of the motion ability of the said individual joint due to the implantation of the artificial joint that has an artificial cartilage of the present invention manufactured for the said individual joint.

In the above method, the said continuous measurement is made during daily living, wherein, the said daily living is defined including the activities lightest to heaviest performed by the said individual, such as resting, (or, sleeping), general daily living activities, and the heaviest sport that the individual can perform at the time deciding to manufacture the said artificial cartilage.

Next, Method A3 is performed: searching and finding a piezoelectric material according to the said corrected range of intra-articular dynamic pressure and said range of the level of Joint-Electricity required of the said individual joint, wherein the piezoelectric material has the material properties at least of: the range of piezoelectric reactivity of the said piezoelectric material must be wider than the said corrected range of intra-articular dynamic pressure, and the range of the level of the electricity the said piezoelectric material generates under the said corrected range of intra-articular dynamic pressure must be wider than the said range of the level of Joint-Electricity required.

More essentially, the piezoelectric material that is so found and meets the above requirement must be a biocompatible material, so that the artificial cartilage does not cause harm to the human body and will not be rejected by the human body. The material property of biocompatibility is essential for all medical or bioengineering devices that are implanted into the human body. And thus, the requirement of the material property of biocompatibility for making an artificial cartilage is known in the related art, and is not invented by the present invention.

Then, Method A4 is performed: forming the said individual artificial cartilage of the said individual artificial joint for the said individual joint of the said individual with the said piezoelectric material.

In the present invention, the said individual artificial cartilage is manufactured following the measurements and estimations of the data those are related to the said individual artificial joint of an individual joint. Thus, the said individual artificial cartilage is manufactured aiming the said individual artificial joint for the said individual joint, and being based on the said individual artificial joint for the said individual joint.

Referring to FIG. 2D, the artificial cartilage forming method A4 can be Method A41: the artificial cartilage forming method A41 includes, the said piezoelectric material is formed into the said individual artificial cartilage having the individualized shape of the said individual artificial cartilage according to the structure, shape, and size of the said individual artificial joint of the said individual joint, wherein, the said individualized shape of the said artificial cartilage has a smooth joint-surface, and wherein, the said smooth joint-surface is further made to be extremely smooth as of nanometer scale.

Forming the said artificial cartilage according to the structure and shape of the artificial joint of the individual joint of the individual and the size of the individual artificial joint as mentioned above is for the purpose of having the artificial cartilage not interfering with every part of the artificial cartilage receiving a dynamic pressure in a correct direction and a normal range after the artificial cartilage has been implanted in the artificial joint with the proper surgery, which has been defined in paragraph [0016].

The joint-surface as smooth as of nanometer scale as mentioned above helps reduce potential mechanical abrasion inside the joint, allowing the artificial cartilage not to easily wear out during use thereof.

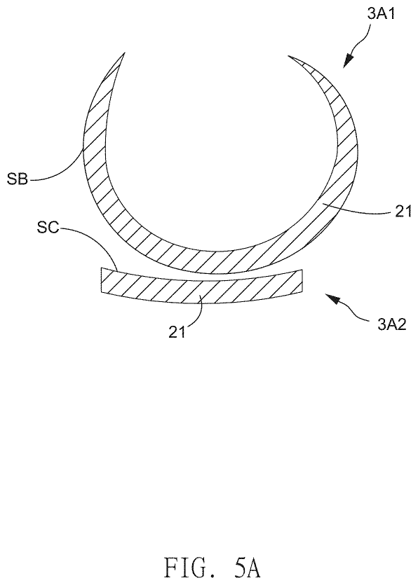

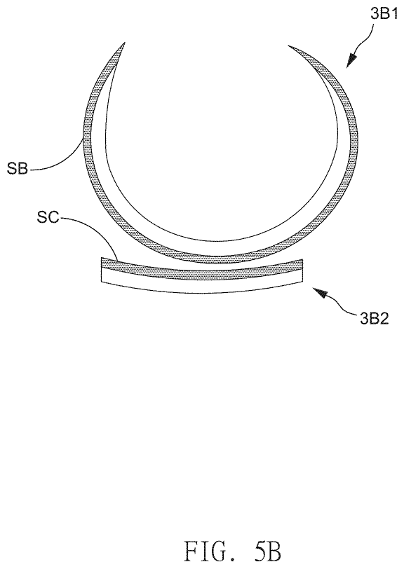

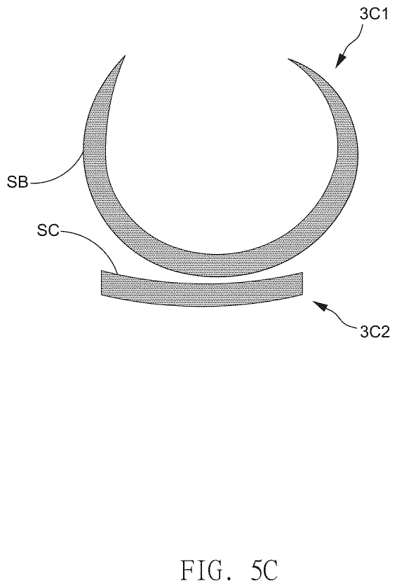

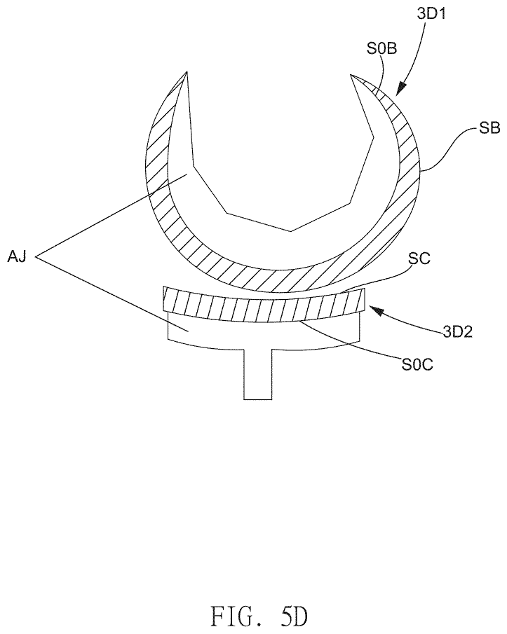

Optionally, the forming method A41 for the artificial cartilage of an individual artificial joint for an individual joint of an individual according to the present invention can be Method A41a: processing the said piezoelectric material into the fibers having a diameter of nanometer size, and forming the said fibers into the said artificial cartilage having the individualized shape of the said individual artificial cartilage according to the structure, shape, and size of the said individual artificial joint, wherein the said individualized shape of the said artificial cartilage has a smooth joint-surface, and through this method, an extremely smooth joint-surface is obtained. The artificial cartilages 3A1, and 3A2 so manufactured are schematically illustrated in sagittal plane in FIG. 5A, and the artificial cartilages 3A1, and, 3A1, each have a joint-surface SB, and, SC, as smooth as of nanometer scale, respectively.

Optionally, the forming method A41 for the artificial cartilage of an individual artificial joint for an individual joint of an individual according to the present invention can be Method A41b: forming the said piezoelectric material into the artificial cartilage having the individualized shape of the said individual artificial cartilage according to the structure, shape, and size of the said individual artificial joint, wherein the said individualized shape of the said artificial cartilage has a smooth joint-surface, and further coating the said piezoelectric material that has been made to be of nanometer size on the said smooth joint-surface in order to have it as smooth as of nanometer scale. The artificial cartilages 3B1, 3B2 so manufactured are schematically illustrated in FIG. 5B, and the artificial cartilages 3B1, 3B2 each have their joint-surfaces SB, and SC extremely smooth as of nanometer scale.

Optionally, the forming method A41 for the said artificial cartilage of an individual artificial joint for an individual according to the present invention can be Method A41c: processing the said piezoelectric material into the particles of nanometer size, and forming the said particles into an artificial cartilage having the individualized shape of the said individual artificial cartilage according to the structure, shape, and size of the said individual artificial joint, wherein the said individualized shape of the said artificial cartilage has a smooth joint-surface, and wherein, the said process has made the said smooth joint-surface as smooth as of nanometer scale. The artificial cartilages 3C1, 3C2 so manufactured are schematically illustrated in FIG. 5C, and the artificial cartilages 3C1, 3C2 each have a joint-surface SB, and SC as smooth as of nanometer scale.

Optionally, the forming method A41 for the artificial cartilage of an individual artificial joint for an individual according to the present invention can be Method A41d: making the said piezoelectric material to be of nanometer size, and carrying out coating with the said nanometer-size material in multiple layers on a primary joint-surface of the said individual artificial joint to form the individualized shape of the said artificial cartilage according to the structure, shape, and size of the said individual artificial joint, wherein the said individualized shape of the artificial cartilage has a smooth joint-surface. Wherein, the said process has simultaneously made an extremely smooth joint-surface as of nanometer scale, SB, and SC, forming the said artificial cartilage in its own individualized shape, and fixing the said artificial cartilage to the said individual artificial joint. This method can only be applied before the surgery of implanting the artificial joint.

The term of primary joint-surface S0B, or S0C as used herein refers to surfaces that are moved toward each other during joint motion in an artificial joint or a site of an end of a bone in a natural joint to which a cartilage is connected to or an artificial cartilage is fixed to. However, the primary joint-surface of an artificial joint can be different from that of a natural joint in shape.

The artificial cartilage manufactured and formed according to Method A41d is applied, as illustrated in the artificial cartilage examples 3D1, 3D2 shown in FIG. 5D, to coating on primary joint-surfaces S0B, S0C. However, the artificial joint shown in FIG. 5D can be of the shapes of all types of artificial joint.

The artificial cartilage according to the present invention is not limited to application to a specific cartilage of any specific joint or an artificial joint of any specific shape, but must follow an individual artificial joint of an individual joint.

Since forming the said artificial cartilage has been based on the structure, shape, and size of the individual artificial joint for the individual joint to have an individualized shape that fits to the said individual artificial joint of the said individual joint, so as not to interfere with the artificial cartilage receiving a range of intra-articular dynamic pressure in a correct direction and a normal range after proper surgery and to generate a sufficient amount of Joint-Electricity.

The present invention also provides a second type of artificial cartilage, which is manufactured with the second method for manufacturing an artificial cartilage to be utilized in an individual artificial joint of an individual joint of an individual. It is made according to the present invention, namely being manufactured with the methods shown in FIGS. 2C and 2D. The steps of manufacturing are shown in FIG. 2C, which is followed by Method of A41, or A41a, or A41b, or A41c, or A41d to manufacture the said artificial cartilage. The examples 1B, and 1C in FIG. 3B, or 3A1, and 3A2 in FIG. 5A, or, 3B1, and 3B2 in FIG. 5B, or 3C1 and 3C2 in FIG. 5C, or 3D1, and 3D2 in FIG. 5D. Other than that showing in FIG. 5D, they can be fixed to the said artificial joint before surgery or can be implanted in an artificial joint AJ of an individual joint during surgery.

The way of using the artificial cartilage of the present invention in an artificial joint is carrying out implantation in an artificial joint of an individual joint with proper surgery described in paragraph [0016]. Except the artificial cartilage that is manufactured with the method of A41d, which must be formed on the individual artificial joint before surgery, the artificial cartilage that is formed with other examples of manufacturing method, such as A41, A41a, A41b, A41c can be fixed to an individual artificial joint before surgery and can also be fixed to the artificial joint during surgery. The term surgery as used here refers to a surgical operation of implanting of an individual artificial joint. The term proper surgery as used here refers to that known to the surgeons who are familiar with artificial cartilage implanting surgery or artificial joint implanting surgery and is not included in the scope of patent protection of the present invention.

In summary of the embodiments described above, based on Joint-Electricity THEORY created by the present inventor, the present invention discloses, in this application, two methods for manufacturing an artificial cartilage and two types of artificial cartilage manufactured with each of the said two methods. In the manufacturing method according to the present invention, the range of piezoelectric reactivity of the artificial cartilage is made of a piezoelectric material that is determined according to an estimation of the range of the intra-articular dynamic pressure of the individual joint to be implanted, and the said piezoelectric material has a range of the level of the electricity generated under the said range of intra-articular dynamic pressure being wider than the range of the level of Joint-Electricity required to be generated by the said individual joint after the implanting surgery. Thus, the artificial cartilage manufactured with the said method, and the artificial joint that is equipped with the said artificial cartilage, during their usage (after the implanting surgery) could persistently and effectively reflect the dynamic pressure in the said individual joint to continuously cause a piezoelectricity effect in order to persistently generate Joint-Electricity, and the level of Joint-Electricity so generated meets the need of the joint of the individual.

The individual artificial cartilage manufactured according to the present invention, for that to be utilized in implanting into an individual natural joint and that an individual artificial joint of an individual joint, after implantation, both continuously generate a sufficient amount of Joint-Electricity in daily living, and thus, may help improve the situation of pain of the joint, muscle strength, and speed the recovery of active motion ability following the surgery. Joint-Electricity generated by the artificial cartilage may also help extend the service life of an artificial cartilage or an artificial joint.

In summary, the above provides just preferred, and feasible, embodiments of the present invention, and is not intended to limit the scope of patent protection of the present invention. Equivalent increases made according to the contents of the specification and drawings of the present invention are regarded as being included in the scope of patent protection of the present invention.

1. A method for manufacturing an artificial cartilage, the method being for manufacturing an artificial cartilage that is to be implanted to an individual joint for an individual, the method comprising:
making continuous measurement on the intra-articular pressure of the contralateral joint of the said individual joint during daily living for deciding the range of the said intra-articular pressure in order to make an estimation of the range of dynamic pressure inside the contralateral joint that acts on the joint-surface of the cartilage of the contralateral joint, and, based on the said range, making an estimation of the range of intra-articular dynamic pressure that acts on the joint-surface of the said artificial cartilage after the said artificial cartilage is implanted;
making continuous measurement on the level of Joint-Electricity generated by the contralateral joint of the said individual joint during daily living for deciding the range of the said level, and, based on the said range, making an estimation of the range of the level of Joint-Electricity that is required to be generated by the said artificial cartilage after the said artificial cartilage is implanted into the said individual joint, the said range is simply called range of the level of Joint-Electricity required;
searching and finding a piezoelectric material according to the said range of intra-articular dynamic pressure, and said range of the level of Joint-Electricity required, wherein the piezoelectric material has the material properties at least of: the range of piezoelectric reactivity of the said piezoelectric material must be wider than the said range of intra-articular dynamic pressure, and the range of the level of electricity the said piezoelectric material generates under the said range of intra-articular dynamic pressure must be wider than the said range of the level of Joint-Electricity required; and
forming the said individual artificial cartilage of the said individual joint with the said piezoelectric material.

2. The method for manufacturing the artificial cartilage according to claim 1, wherein a method for forming the individual artificial cartilage for the said individual joint with the said piezoelectric material includes, the said piezoelectric material being formed into the said artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface is further made to be extremely smooth as of nanometer scale.

3. The method for manufacturing the artificial cartilage according to claim 2, wherein a method for forming the individual artificial cartilage for the said individual joint includes making the said piezoelectric material into the fibers having a diameter of nanometer size, and forming the said fibers into an artificial cartilage having the individualized shape of the said individual artificial cartilage according to the structure, shape, and size of the said individual joint, wherein the said individualized shape of the said artificial cartilage has a smooth joint-surface, and wherein, the said smooth joint-surface has become extremely smooth as of nanometer scale.

4. The method for manufacturing the artificial cartilage according to claim 2, wherein the method for forming the individual artificial cartilage for the said individual joint includes forming the said piezoelectric material into an artificial cartilage having the individualized shape of the said individual artificial cartilage according to the structure, shape, and size of the said individual joint, wherein the said individualized shape of the said artificial cartilage has a smooth joint-surface, and then the said smooth joint-surface being further coated with the said piezoelectric material that has been processed to be of nanometer size for making the said joint-surface extremely smooth as of nanometer scale.

5. The method for manufacturing the artificial cartilage according to claim 2, wherein the method for forming the said individual artificial cartilage for the said individual joint includes forming the said piezoelectric material into an artificial cartilage having the individualized shape of the said individual artificial cartilage according to the structure, shape, and size of the said individual joint, wherein the said individualized shape of the said artificial cartilage has a smooth joint-surface, and then the said smooth joint-surface being further coated with the said piezoelectric material that has been processed to be of nanometer size for making the said joint-surface extremely smooth as of nanometer scale.

6. An artificial cartilage, the said artificial cartilage being manufactured with the method according to claim 1.

7. An artificial cartilage, the said artificial cartilage being manufactured with the method according to claim 2.

8. A method for manufacturing an artificial cartilage, the said method being for manufacturing an artificial cartilage that is to be implanted or fixed to an individual artificial joint of an individual joint for an individual, the method comprising:
making continuous measurement on the intra-articular pressure of the contralateral joint of the said individual joint during daily living for a range of the said intra-articular pressure in order to make an estimation of a range of dynamic pressure inside the said contralateral joint that acts on the joint-surface of cartilage of the said contralateral joint, based on the said range, making an estimation of a range of intra-articular dynamic pressure that acts on the joint-surface of the said individual artificial cartilage after the said individual artificial cartilage has been implanted to the said individual joint, and applying a force transmission correction parameter to correct the said range of intra-articular dynamic pressure to obtain a corrected range of intra-articular dynamic pressure, which is the estimation of the range of dynamic pressure inside the said individual artificial joint that acts on the joint-surface of the said artificial cartilage after the said artificial joint is implanted, wherein the force transmission correction parameter is determined according to the structure and material of the said individual artificial joint;
making continuous measurement on the level of Joint-Electricity generated by the contralateral joint of the said individual joint during daily living for a range of the said level, and, based on the said range, making an estimation of a range of the level of Joint-Electricity required to be generated by the said individual joint after the said individual artificial cartilage is implanted into the said individual artificial joint, wherein the said range is simply called range of the level of Joint-Electricity required;
searching and finding a piezoelectric material according to the said corrected range of intra-articular dynamic pressure and said range of the level of Joint-Electricity required of the said individual joint, wherein the piezoelectric material has the material properties at least of: the range of piezoelectric reactivity of the said piezoelectric material must be wider than the said corrected range of intra-articular dynamic pressure, and the range of the level of electricity generated by the said piezoelectric material under the said corrected range of intra-articular dynamic pressure must be wider than the said range of the level of Joint-Electricity required; and
forming the said individual artificial cartilage of the said individual artificial joint for the said individual joint of the said individual with the said piezoelectric material.

9. The method for manufacturing the artificial cartilage according to claim 8, wherein a method for forming the artificial cartilage for an individual artificial joint of an individual joint for an individual includes, the said piezoelectric material being formed into the said individual artificial cartilage having the individualized shape of the said individual artificial cartilage according to the structure, shape, and size of the said individual artificial joint of the said individual joint, wherein, the said individualized shape of the said artificial cartilage has a smooth joint-surface, and wherein, the said smooth joint-surface is further made to be extremely smooth as of nanometer scale.

10. The method for manufacturing the artificial cartilage according to claim 9, wherein the method for forming the artificial cartilage for an individual artificial joint of an individual joint for an individual includes processing the said piezoelectric material into the fibers having a diameter of nanometer size, and forming the said fibers into the said artificial cartilage having the individualized shape of the said individual artificial cartilage according to the structure, shape, and size of the said individual artificial joint, wherein the said individualized shape of the said artificial cartilage has a smooth joint-surface, and through this method, an extremely smooth joint-surface as of nanometer scale is also obtained.

11. The method for manufacturing the artificial cartilage according to claim 9, wherein the method for forming an artificial cartilage for an individual artificial joint of an individual joint for an individual includes forming the said piezoelectric material into the artificial cartilage having the individualized shape of the said individual artificial cartilage according to the structure, shape, and size of the said individual artificial joint, wherein the said individualized shape of the said artificial cartilage has a smooth joint-surface, and further coating the said piezoelectric material that has been made to be of nanometer size on the said smooth joint-surface in order to have it as smooth as of nanometer scale.

12. The method for manufacturing the artificial cartilage according to claim 9, wherein the method for forming the artificial cartilage for an individual artificial joint of an individual joint for an individual includes processing the said piezoelectric material into the particles of nanometer size, and forming the said particles into an artificial cartilage having the individualized shape of the said individual artificial cartilage according to the structure, shape, and size of the said individual artificial joint, wherein the said individualized shape of the said artificial cartilage has a smooth joint-surface, and wherein, the said process has made the said smooth joint-surface as smooth as of nanometer scale.

13. The method for manufacturing the artificial cartilage according to claim 9, wherein the method for forming the artificial cartilage for an individual artificial joint of an individual joint for an individual includes making the said piezoelectric material to be of nanometer size, and carrying out coating with the said nanometer-size material in multiple layers on a primary joint-surface of the said individual artificial joint to form the individualized shape of the said artificial cartilage according to the structure, shape, and size of the said individual artificial joint, wherein the said individualized shape of the artificial cartilage has a smooth joint-surface, wherein, the said process has simultaneously made an extremely smooth joint-surface as of nanometer scale, and wherein, primary joint-surface as used herein refers to surfaces in an artificial joint that are moved toward each other during joint motion.

14. An artificial cartilage, the said artificial cartilage being manufactured with the method according to claim 8.

15. An artificial cartilage, the said artificial cartilage being manufactured with the method according to claim 9.
